(19) United States
(12) Patent Application Publication
(10) Pub. No.: US 2007/0183988 A1
(43) Pub. Date: Aug. 9, 2007

(54) Tooth whitening delivery system

(75) Inventors: William E. Prosise, Ramsey, NJ (US); Jonathan B. Levine, Purchase, NY (US)

Correspondence Address: H. JAY SPIEGEL, P.O. BOX 11, Mount Vernon, VA, 22121, US

(73) Assignees: GoSMILE, INC; ISP Investments. Inc.

(21) Appl. No.: 11/346,263

(22) Filed: Feb. 3, 2006

(51) Int. Cl. A61K 8/22; A61K 8/81

(52) U.S. Cl. 424/53

(57) ABSTRACT

A tooth whitening delivery system including a single-dose applicator and a liquid, substantially anhydrous tooth whitener composition of predetermined viscosity contained in said applicator for delivery of a tooth-whitening amount of active whitening ingredient from said applicator onto teeth, with effective bioadhesion and retention thereof, and, thereupon, sufficient penetration of said ingredient through the tooth enamel providing efficacious bleaching of stained teeth, comprising, by weight,

(a) 10% to 75% of a complex which is (i) a mixture of 78 to 90% of water soluble polyvinylpyrrolidone (PVP) having K-values of K-12 to K-120 and (ii) 10 to 22% of H₂O₂; releasing 1 to 20% of active H₂O₂ from said complex onto a tooth surface; (b) 0 to 20% of PVP K-60 to K-90, said PVP being present in said composition at a K-value of the PVP in said complex of less than 30, and/or the amount of PVP therein is less than 20%, and (c) 30 to 90% of a substantially anhydrous solvent, said composition having a Brookfield viscosity of 75 to 300 cps, an in vitro bioadhesion/retention on hydroxyapatite (HAP) of at least 10% after 5 minutes while immersed in water, and a bleaching efficacy defined by ΔE values of at least 5 in the CIE (L*a*b*) color scale.

### CROSS-REFERENCE TO RELATED U.S. PATENT APPLICATIONS

This application is related to U.S. Patent Application Publication No. US 2003/0203338A1, published Oct. 30, 2003; and U.S. patent application Ser. No. 11/168,163, filed Jun. 28, 2005.

### BACKGROUND OF THE INVENTION

1. Field of the Invention

The present invention relates to a method of applying a composition, and, more particularly, to a method of applying a defined tooth whitening composition of predetermined viscosity from a single-dose applicator and the tooth whitening delivery system used to practice the method.

2. Description of the Prior Art

Since the early 1980s, the tooth whitening industry has grown from a scenario in which one could only receive tooth whitening treatments in a dental office to the scenario that exists today in which numerous competitors sell over-the-counter tooth whitening systems including delivery systems such that the average consumer can whiten their teeth in their own home without medical supervision.

Numerous delivery systems have become popular and well known including those employing strips coated with the tooth whitening substance, trays filled with tooth whitener, tooth whiteners incorporated into toothpaste, and other paint-on applicators such as those analogous to felt tip pens, and others using brushes with bristles and foam-type brush applicators. Over-the-counter whiteners today fall into three categories—trays and gels, strips and paint-on whiteners.

Tray and gel systems are designed for the user to fill the flexible tray with the whitening formulation and this is placed on the teeth. The gel extrudes out of the tray on the gingival and the mucosa with a great chance of soft tissue irritation. Strips are placed on the teeth with a good portion of the strip laying on the gingival area and also causing irritation.

A successful tooth whitening composition requires that the following parameters be included:

(1) a sufficient amount of activated active ingredient, e.g. H₂O₂ present in and delivered by the composition;

(2) an enhanced degree of retention of the composition onto teeth; i.e. its bioadhesion, which is determined by the ratio of its cohesive energy to surface energy;

(3) a favorable degree of penetration of the composition into the tooth enamel;

(4) a suitable viscosity to deliver the composition by selected desirable delivery means; and,

(5) efficacy of bleaching of stained teeth enamel by the composition.

Accordingly, it is the object of this invention to provide a tooth whitener composition and delivery system having, in combination:

(1) a predetermined concentration of active H₂O₂ in a film-forming complex; which provides for;

(2) effective bioadhesion/retention of the composition on teeth;

(3) advantageous penetration of the composition into the tooth enamel;

(4) a predeterminable viscosity to deliver the composition onto teeth by a suitable delivery means; and

(5) excellent bleaching action on stained teeth.

The following patents illustrate active tooth whitening substances and delivery systems for such substances: U.S. Pat. Nos. 5,077,047, 5,108,742; 5,122,370; 5,130,124; 5,183,901; 5,190,749; 5,206,385; 5,312,619; 5,989,569; 6,669,930; 6,770,266 and 6,893,629.

U.S. Pat. No. 5,098,297 to Chari et al. discloses an apparatus for application of a tooth desensitizing composition. However, Chari et al. fail to teach or suggest tooth whitening; instead, they disclose the use of a swab-type applicator that retains and dispenses a tooth desensitizer onto the teeth. Chari et al. disclose a separate filter disc designed to prevent shards of glass from an ampule containing the desensitizing solution from escaping into the mouth of the patient. The present method differs from the teachings of Chari et al. in several respects. First, the present invention contemplates application of a defined tooth whitening composition of a predetermined viscosity rather than a tooth desensitizing composition. Second, the present invention contemplates evacuating the interior of the glass ampule containing a monodose of the tooth whitening composition so that its shelf life is extended. Third, the present invention contemplates use of a brush to dispense the tooth whitening composition with the brush doubling as the filter preventing glass shards from entering the mouth of the patient. Furthermore, the tooth whitening composition used herein is more viscous than the tooth desensitizing composition taught by Chari et al. Accordingly, the wad-type applicator of Chari et al. would not be suitable for use in dispensing the tooth whitening composition of the present invention.

### BRIEF DESCRIPTION OF THE DRAWINGS

FIG. 1 is a plot of % Retention vs. Immersion Time for the tooth whitening composition of the invention on hydroxyapatite.

FIG. 2 shows a side view of an applicator with the cardboard protector thereof shown in phantom.

FIG. 3 shows a side view of the cardboard protector with the internal chamber thereof shown in phantom.

FIG. 4 shows a side view of the applicator brush of the present invention.

FIG. 5 shows an end view of the brush applicator.

FIG. 6 shows a magnified view of a portion of the view of FIG. 4 to show further details.

### SUMMARY OF THE INVENTION

What is described herein is a liquid, substantially anhydrous tooth whitener composition of predetermined viscosity for effective delivery of a tooth-whitening amount of active whitening ingredient onto teeth from a single-dose applicator, which exhibits, excellent bioadhesion and retention of said ingredient on teeth, advantageous penetration of said ingredient through tooth enamel, and effective bleaching of stained teeth, comprising, by weight,

(a) 10% to 75% of a complex which is (i) a mixture of 78 to 90% of water soluble polyvinylpyrrolidone (PVP) having a molecular weight corresponding to K-12 to K-120 values, and (ii) 10 to 22% of H₂O₂; providing 1 to 20% of active H₂O₂ onto a tooth surface;

(b) 0 to 20% of PVP K-60 to K-90, said PVP being present in said composition when the PVP in said complex has a K-value of less than 30, and/or the amount of PVP therein is less than 20%, and

(c) 30 to 90% of a substantially anhydrous solvent,

said composition having, in combination, a Brookfield viscosity of 75 to 300 cps, an in vitro bioadhesion and retention on hydroxyapatite of at least 10% after 5 minutes while immersed in water, and a bleaching efficacy defined by ΔE values of at least 5 in the CIE (L*a*b*) color scale.

Suitably, the tooth whitener composition of the invention includes (a) PVP K-30 to K-90. When present, the PVP in (b) is suitably K-90.

Alternatively, the tooth whitener composition herein includes PVP K-90 in (a), and (b) is absent.

Preferably the amount of the complex (a) in the composition is 20 to 60% and (b) is 0.1-10%.

Preferably the solvent in the tooth whitener composition is ethanol in an amount of 65 to 80 wt. % of the composition.

Preferably, the tooth whitener composition of the invention has a viscosity of 75 to 300 cps, most preferably 125 cps.

The inventive method includes in one embodiment providing an ampule with the defined tooth whitening composition which is vacuum sealed within a chamber of the ampule. The ampule may be contained within a cylindrical plastic sleeve closed at one end and enclosed at its other open end by the proximal termination of a brush-type applicator. An adhesive such as glue may be used to adhere the periphery of the brush to the inner surfaces of the plastic cylinder. The bristles preferably have circular cross-sections so that when they are tightly packed together and adhered to the plastic cylinder, the interstices between adjacent bristles are sufficiently large enough to permit flow through of the tooth whitening composition, while being sufficiently small enough to prevent shards of glass from the ampule, when the ampule is made of glass and is broken open, to pass through the brush. The bristles are used to apply the tooth whitening composition to teeth where it is retained for a sufficient time to whiten the teeth and then it is rinsed off with water.

Other embodiments of the present invention incorporate use of any desired single dose applicator for tooth whitener. Examples of such applicators include a dipstick-type applicator including a single-dose chamber into which a soft liquid absorbent applicator tip is inserted to absorb a desired quantity of tooth whitener to be applied to the user's teeth, as well as applicators wherein the container for the single dose of tooth whitener also comprises the applicator itself. In such an example, an overcap may initially cover a porous applicator mounted over a sealed opening to a chamber containing tooth whitener. The overcap includes a downwardly depending inner pin that may pierce the seal through the porous applicator to cause the tooth whitener to be absorbed by the applicator and then applied to the user's teeth. Any other single dose applicator may suitably be used in accordance with the teachings of the present invention.

### DESCRIPTION OF THE INVENTION

1. Amount of Activated H₂O₂

In one embodiment of the invention, the complex in the tooth whitening composition is available as the commercial product PEROXYDONE® (ISP), which is made up of, by weight, 80-83%, of water soluble polyvinylpyrrolidone (PVP), e.g. K-15/K-30/K-90, and 17-20%, by weight, hydrogen peroxide (H₂O₂).

This PVP-H₂O₂ complex, with or without added (b) PVP, e.g. Plasdone® K-90 (ISP), to increase its viscosity, is mixed with a suitable proportion of a solvent, e.g. absolute ethanol. When applied to the surfaces of the teeth of the user, the ethanol immediately begins to evaporate resulting in a marked increase in the concentration of H₂O₂ thereby enhancing the whitening power of the formulation.

Typical tooth whitening compositions of the invention are shown in Tables 1 and 2 below.

TABLE 1

| | EXAMPLE NO. | | | | | | | | |
|---|---|---|---|---|---|---|---|---|---|
| Ingredient | 1 | 2 | 3 | 4 | 5 | 6 | 7 | 8 | 9 |
| Peroxydone ® K-30 | 23.3 | 27.77 | 40.0 | 37.5 | 35.0 | 23.3 | 27.8 | 40.0 | 23.3 |
| % H₂O₂ | 4.2 | 5.1 | 8.0 | 7.5 | 7.0 | 4.7 | 5.6 | 7.8 | 4.3 |
| Plasdone ® K-90 | 5.00 | 3.75 | — | — | — | 3.50 | 2.50 | — | 4.75 |
| Ethanol (Abs.) | 71.2 | 67.98 | 59.5 | 62.0 | 64.5 | 62.9 | 69.1 | 59.4 | 71.35 |
| Brookfield Viscosity, cPs | 246 | 244 | 280 | 172 | 120 | 117 | 124 | 217 | 206 |

TABLE 2

| | EXAMPLE NO. | | | |
|---|---|---|---|---|
| Ingredient | 10 | 11 | 12 | 13 |
| Peroxydone ® K-30 | 27.78 | 17.50 | 20.00 | 40.00 |
| Peroxydone ® K-90 | — | 7.50 | 5.00 | — |
| % H₂O₂ | 5.3 | 5.0 | 4.8 | 7.9 |
| Plasdone ® K-90 | 3.50 | — | — | 5.00 |
| Ethanol (Abs.) | 68.12 | 74.58 | 74.5 | 54.5 |
| Brookfield Viscosity, cPs | 195 | 272 | 171 | 179 |

TABLE 3

| Example No. | Ingredients |
|---|---|
| 14 | 25% Peroxydone K-30 in Ethanol (18.7% H₂O₂) |
| 15 | 25% Peroxydone K-90 in Ethanol (17.5% H₂O₂) |
| 16 | 12.5% Peroxydone K-30 and 12.5% Peroxydone K-90 |
| | in Ethanol |
| 17 | 50% Peroxydone K-30 in Ethanol |
| 18 | 30% Peroxydone K-30 in PEG 400 |

2. Viscosity of Composition

As is shown in Tables 1 and 2, when the PVP ingredient in the complex includes PVP having a K-value of K-90, and the complex is present in an amount of at least 15% of the composition, the viscosity of the composition increases substantially. On the other hand, when the K-value of the PVP in the complex is K-30 or lower, and its amount therein is 40% or lower, the viscosity of the composition is less than 300 cps. A suitable viscosity for delivery from the ampule applicator is about 75 to 300 cps, preferably about 100 to 200 cps.

3. Bioadhesive/Retention of Composition on Teeth

The relative retention capacity of a test formulation on tooth surfaces can be measured by weight difference from the surface of a hydroxyapatite (HAP) plate that has been coated with the formulation and immersed in artificial saliva. An in-vitro test for formula weight retention on HAP is described below.

These in-vitro tests for formulation efficacy can be used to collectively determine the relative performance of paint-on teeth bleaching compositions.

Formula Retention Test

a) Accurately tare HAP-coated glass plate to 3 decimal places.
b) Paint HAP surface with a single coat of product, wait 30 seconds, and reweigh (3 dec.).
c) Place coated HAP in one liter of artificial saliva for 0, 1, 2 and 5 minutes time.
d) Remove each slide after its specified time, dry to constant weight, and weigh.
e) Determine initial product weight, and product weight after 1, 2 and 5 minutes submersion in artificial saliva.
f) Calculate percent retained on HAP versus time.

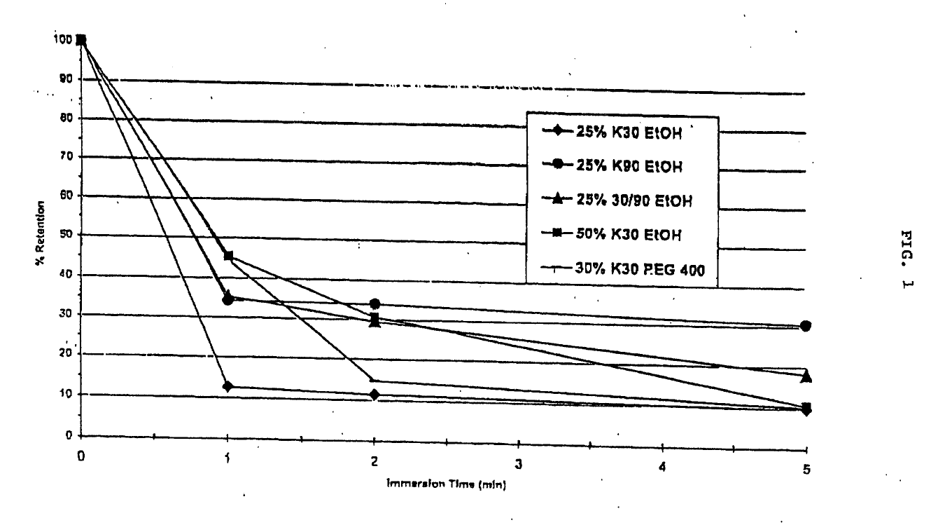

FIG. 1 presents the percent retention on hydroxyapatite (HAP) versus immersion time of the teeth whitening formulations of the invention (Examples 14-18).

After five minutes immersion time, the 25% K-90 EtOH formulation is the best retained on HAP. The blend of K-90 and K-30 also exhibits effective retained time on HAP (at five minutes), as did the 50% K-30 EtOH formulation.

4. Bleaching Efficacy

The stain bleaching efficacy of the teeth whitening products compositions of the invention is determined by the rate of peroxide bioadhesion into the teeth and retention of the vehicle on the tooth surface. Two recent in-vitro methods to measure bleaching activity of such compositions were developed by Colgate-Palmolive Company and published in a poster session at the 2004 meeting of the Intl. Assoc, for Dental Research*.
*Subramanyam, R.; Cameron, R; Colgate Palmolive Co.; “Comparative Delivery of Bleaching Agent from At-home Whitening Products”; Poster #3527; Presented at IADR Annual Meeting; Honolulu, Hi.; Mar. 10-13, 2004

These methods are based on a dye compound that acts as a surrogate for teeth stain on the exterior of teeth (extrinsic stain) or within the teeth (intrinsic stain). The rate of bleaching of a fixed amount of dye can thus indicate the peroxide release rate from a formulation. Additionally, the relative rate of absorption or penetration by the peroxide from the formulations can be determined by the bleaching of a previously dyed porous substance.

Formulation bleaching efficacy also can be determined by measuring the instrumental color difference of stained HAP disks before and after bleaching. A test was developed that measures the L*a*b* color of tea-stained disks before staining, after staining, and after bleaching. Color differences (ΔE) as a result of bleaching with different formulations can be determined mathematically.

Three separate bleaching efficacy tests were developed. The following procedures are presented for each separate test. Five simple Peroxydon®-based formulas of the invention were evaluated for performance in each test method. The hydrogen peroxide content was determined by permanganate titration.

Table 4 lists the averaged L*a*b* color results of each two disk set before staining, after staining and after staining and bleaching with each of the five prototype whitening formulations. ΔE color differences between each variable and no stain were calculated. Additionally, ΔE color differences realized by bleaching were calculated. These are ΔE's between stained disked before and after bleaching.

TABLE 4
B/4 Bleaching

| | Stain | Stain | Stain | Stain | Stain |
|---|---|---|---|---|---|
| Dry | Only | Only | Only | Only | Only |
| L | 80.97 | 81.28 | 79.87 | 79.08 | 81.99 |
| A | 4.29 | 4.19 | 4.56 | 4.33 | 4.37 |
| B | 9.15 | 9.09 | 8.66 | 8.94 | 9.04 |
| E vs | 19.388 | 19.073 | 20.174 | 20.902 | 18.522 |
| No Stain | | | | | |

After Bleaching

| | 25% K-30 | 25% K-90 | 25% 30/90 | 50% K-30 | 30% K-30 |
|---|---|---|---|---|---|
| Dry | EtOH | EtOH | EtOH | EtOH | PEG 400 |
| L | 89.69 | 84.11 | 81.54 | 91.71 | 89.49 |
| A | 0.98 | 2.51 | 4.11 | 0.15 | 1.28 |
| B | 4.41 | 15.52 | 18.88 | 2.76 | 3.34 |
| E vs | 8.938 | 20.646 | 25.106 | 6.242 | 8.711 |
| No Stain | | | | | |
| Δ E - Stain | 10.464 | 7.218 | 10.367 | 14.668 | 9.918 |
| Removal by | | | | | |
| Bleaching | | | | | |

HAP Disk Bleaching Test
a) Tea Stain Solution Preparation—Ten (10) Lipton tea bags (25.4 g including paper) were placed in 215 g D.I. water at 195 F. and held for 25 minutes. Bags were removed and stain solution was immediately used.
b) Formation of Pellicle and Uptake of Stain onto HAP Disks—Each disk was covered with 2 g of 1% mucin supernatant and incubated overnight (20 hours) in a test tube at 37° C. with gentle shaking using a vortex mixer at about a 400 speed. This forms a mucin coating on the disk that simulates a salivary pellicle.
c) Staining Disks—Each disk was removed from the mucin supernatant and rinsed by dipping in a large excess of D.I. water three times. All disks, except two (unstained), were individually placed in 2 ml of tea stain concentrate in test tubes, and incubated at 37° C. for one (1) hour with gentle shaking at a speed of 300. The disks were each removed from the stain, rinsed three (3) times by dipping in excess D.I. water and air-dried overnight.
d) All disks were individually numbered and separated into two disk sets. The color of each disk was measured by Minolta Spectrophotometer over the entire visual spectrum and by L*, a*, b*. Colors were averaged for each of the two-disk sets.
e) Stained disks were hydrated in artificial saliva for four hours, and placed flat in a petri plate partially submerged in artificial saliva.
f) Disk top surfaces were blotted dry, while partially submerged, and each two-disk set was painted with a single coat of teeth whitener (bleach) and held (covered, partially submerged) for 30 minutes.
g) Disks were removed from artificial saliva, rinsed three times in D.I. water, wiped dry, and placed in a desiccator overnight (20 hours) to dry.
h) The color of each bleached disk was measured by Minolta Spectrophotometer (as d above). Colors were averaged for each of the two-disk sets.
i) Average colors were plotted and appropriate ΔE values (mathematical color differences) were calculated. L*, a*, b* color and ΔE values are given below.

### Reflected Color by Hunter Colorimeter

Commission Internationale de l'Eclairage (CIE) uses L*a*b* scale

L is lightness 0=black, 100=pure white

a is yellow (+) and blue (−)

b is red (+) and green (−)

Total Color Difference (ΔE) between a sample and standard (or control)
ΔE=((ΔL)²+(Δa)²+(Δb)²)^{0.5}
Reference: Hunter Associates Laboratory, Reston, Va.

The ampule apparatus includes, in one embodiment, a glass ampule having a monodose of the tooth whitening composition vacuum sealed therein. The ampule is contained within a cylindrical plastic sleeve closed at one end and enclosed at its other open end by the proximal termination of a brush-type applicator. An adhesive such as glue is used to adhere the periphery of the brush to the inner surfaces of the open end of the plastic sleeve.

The bristles preferably have circular cross-sections so that when they are tightly packed together and peripheral bristles are adhered to the plastic cylinder, interstices between adjacent bristles are sufficiently large enough to permit flow through of the tooth whitening composition while being sufficiently small enough to prevent shards of glass from the ampule, when made of glass, when broken open, to pass therethrough. The ampule may be made of any suitable material, including various plastics, so long as it can be broken open when desired to release the tooth whitener contained therein.

A cardboard sleeve surrounds the plastic cylinder to protect the user from the glass ampule when broken.

The bristles of the brush applicator are sized and configured to permit brushing between teeth into the tooth enamel. It is in these areas where stains often accrue., namely, the inter-proximal areas of the teeth. Thus, a brush rather than a wad is chosen due to its effectiveness with regard to the anatomy of the teeth that are being treated.

The method of delivering the tooth whitening composition in the ampule apparatus, involves
(a) providing the tooth whitening composition defined above vacuum sealed within the glass ampule;

(b) placing the ampule within a plastic cylinder having one end closed and the other end open;

(c) sealing the open end of the plastic cylinder by the proximal end of a brush through the use of adhesive applied therebetween;

(d) providing the brush with bristles that are tightly adjacent one another and have spaces therebetween permitting flow of tooth whitening composition therethrough;

(e) placing a protector, preferably made of cardboard, over the plastic cylinder (this step may be omitted where the ampule is not made of glass);

(f) squeezing the glass ampule through the cardboard protector until it shatters, thus releasing the tooth whitening composition into the plastic cylinder;

(g) flowing the tooth whitening composition between the bristles of the brush while the tightly packed bristles act as a filter preventing the shards of glass from the ampule from traveling therebetween;

(h) pulsating the cardboard protector to dispense the tooth whitening composition through the bristles with the bristles being run over the teeth of the user particularly in the inter-proximal areas until the tooth whitening composition is completely applied to the teeth;

(i) adhering the tooth whitening composition to the teeth for a sufficient period of time so that the tooth whitening composition effectively operates to whiten the user's teeth; and,
(j) after a prescribed time period, rinsing the mouth with water to rinse away the tooth whitening composition.

Reference is first made to FIG. 2 in which the apparatus of a first embodiment of the present invention is generally designated by the reference numeral 10 and is seen to include a preferably cylindrical plastic sleeve 11 containing an ampule 13 preferably made of glass in which is vacuum sealed a measured quantity of tooth whitening composition 15, preferably a monodose of that solution.

The plastic cylindrical sleeve 11 has a closed bottom 17 and is upwardly open at 19.

An applicator tip 21 is best seen in FIG. 5 to consist of a plurality of individual bristles 23 (see also FIG. 5) closely packed together and enclosed within the opening 19 of the plastic cylinder 11.

As seen in FIG. 6, the individual bristles 23 are interconnected by a wire-like connector 25 during the manufacturing process thereof and are packed together as shown in FIGS. 5 and 6, so that they include interstices 27 existing between respective adjacent bristles 23.

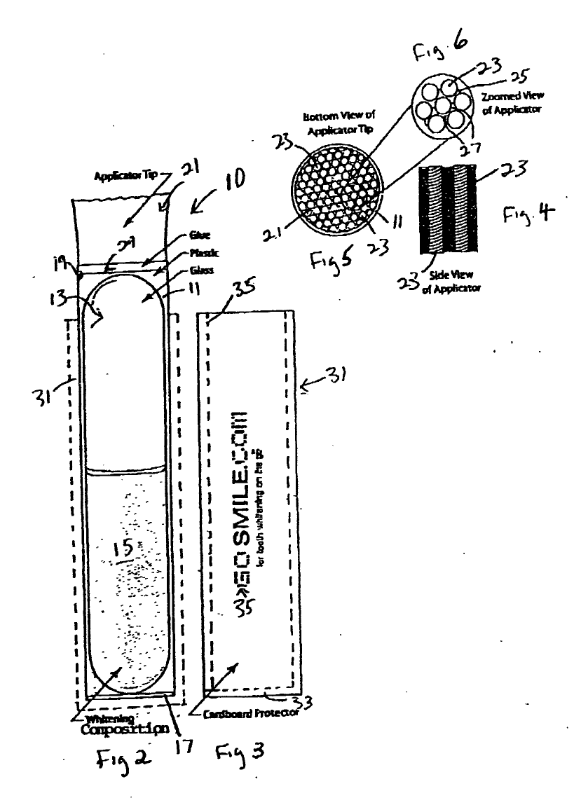

As seen in FIG. 2, a band 29 of glue extends about the inner periphery of the plastic cylinder 11 adjacent the opening 19 thereof and adheres the bristles 23 as shown in FIG. 5 so that they act to close the opening 19 except for the interstices 27. FIG. 4 shows a side view of individual bristles 23 packed together in accordance with the teachings of the present invention.

As seen in FIGS. 2 and 3, a protector 31 preferably made of cardboard or other soft flexible material encloses much of the plastic cylinder 11. For this purpose, the protector 31 has a closed bottom 33 and an upper opening 35 sized to closely receive the outer periphery of the plastic cylinder 11 therein. A logo 35 or other markings may suitably be printed on the protector 31. The protector may be omitted where the ampule is not made of glass.

In the operation of the apparatus 10, in the configuration shown in FIG. 2, the plastic cylinder 11 is squeezed through the protector 31 to break the ampule 11, thereby allowing exit therefrom of the tooth whitening composition 15. The protector 31 is squeezed, thereby compressing the plastic cylinder 11 and causing the tooth whitening composition 15 to exit the plastic cylinder by entering the interstices 27 of the applicator 21. The interstices 27 are small enough to preclude any glass shards from the ampule 13, when made of glass, from traveling through the applicator 21 and into the mouth of the user. The applicator 21 is run over the teeth of the user while the plastic cylinder 11 is being pulsated by rhythmically squeezing the protector 31. Tooth whitening composition 15 is dispensed through the interstices between the bristles 23 and onto the teeth of the user. The brush is specifically designed to allow application of the tooth whitening solution 15 in the inter-proximal area of the teeth, namely, between adjacent teeth which is where stains typically accrue. The predetermined viscosity of 75 to 300 cps of the tooth whitening composition 15 precludes it from washing away until the user actively rinses their mouth. With the tooth whitening composition adhering to the enamel surfaces of the teeth, the teeth are whitened. After a prescribed time period, the mouth is rinsed to rinse away the tooth whitening composition after the teeth have been effectively whitened.

Other embodiments of the present invention incorporate use of any desired single dose applicator for tooth whitener. Examples of such applicators include a dipstick-type applicator including a single-dose chamber into which a soft liquid absorbent applicator tip is inserted to absorb a desired quantity of tooth whitener to be applied to the user's teeth, as well as applicators wherein the container for the single dose of tooth whitener also comprises the applicator itself. In such an example, an overcap may initially cover a porous applicator mounted over a sealed opening to a chamber containing tooth whitener. The overcap includes a downwardly depending inner pin that may pierce the seal through the porous applicator to cause the tooth whitener to be absorbed by the applicator and then applied to the user's teeth. Any other single dose applicator may suitably be used in accordance with the teachings of the present invention.

While the invention has been described with particular reference to certain embodiments thereof, it will be understood that changes and modifications may be made which are within the skill of the art.

What is claimed is:

1. A tooth whitening delivery system comprising a single dose applicator and a liquid, substantially anhydrous tooth whitener composition of predetermined viscosity contained in said applicator for delivery of a tooth-whitening amount of active whitening ingredient from said applicator onto teeth, with effective bioadhesion and retention thereof, and, thereupon, sufficient penetration of said ingredient through the tooth enamel providing efficacious bleaching of stained teeth, comprising, by weight,
(a) 10% to 75% of a complex which is (i) a mixture of 78 to 90% of water soluble polyvinylpyrrolidone (PVP) having K-values of K-12 to K-120 and (ii) 10 to 22% of H₂O₂; releasing 1 to 20% of active H₂O₂ from said complex onto a tooth surface;
(b) 0 to 20% of PVP K-60 to K-90, said PVP being present in said composition at a K-value of the PVP in said complex of less than 30, and/or the amount of PVP therein is less than 20%, and
(c) 30 to 90% of a substantially anhydrous solvent,
said composition having a Brookfield viscosity of 75 to 300 cps, an in vitro bioadhesion/retention on hydroxyapatite (HAP) of at least 10% after 5 minutes while immersed in water, and a bleaching efficacy defined by E values in the CIE (L*a*b*) color scale of at least 5.

2. A tooth whitening delivery system according to claim 1 wherein the PVP in (a) is PVP K-30.

3. A tooth whitening delivery system according to claim 1 wherein the PVP in (b) is PVP K-90.

4. A tooth whitening delivery system according to claim 1 wherein the PVP in (a) is K-90.

5. A tooth whitening delivery system according to claim 1 wherein said amount of solvent in (c) is 65 to 80%.

6. A tooth whitening delivery system according to claim 1 wherein said solvent (c) is ethanol.

7. A tooth whitening delivery system according to claim 1 wherein said composition has a viscosity of 100-200 cps.

8. A tooth whitening delivery system according to claim 7 wherein said composition has a viscosity of 125 cps.

9. A tooth whitening delivery system according to claim 1 wherein the amount of said complex comprises: (a) 20 to 60%, and (b) 0.1-10%.

10. A tooth whitening delivery system according to claim 1 wherein said applicator comprises a tooth whitening applicator including:
i) an ampule containing a tooth whitening composition;
ii) a flexible sleeve with a closed end and an open end and including an internal chamber, said ampule received within said chamber;
iii) said open end being closed by a proximal end of a brush having bristles closely packed together and defining narrow interstices between adjacent bristles, said brush extending beyond said open end of said sleeve; a method of applying said tooth whitening composition including the steps of:
a) breaking said ampule and releasing said tooth whitening composition;
b) conveying said tooth whitening composition through said interstices while said bristles prevent pieces of said ampule from exiting said sleeve;
c) using said brush to apply said tooth whitening composition to teeth.

11. The system of claim 10, wherein said ampule is made of glass.

12. The method of claim 11, wherein said tooth whitening composition is vacuum packed within said ampule.

13. The method of claim 10, wherein said tooth whitening composition is vacuum packed within said ampule.

14. The method of claim 10, wherein said flexible sleeve is made of plastic.

15. The method of claim 10, further including the step of gluing said bristles into said open end of said sleeve.

16. The method of claim 10, wherein said bristles have circular cross-sections.

17. The method of claim 10, wherein said applicator has a protector receiving said sleeve therein.

18. The method of claim 17, wherein said releasing step includes the step of squeezing said sleeve.

19. The method of claim 10, wherein said releasing step includes the step of squeezing said sleeve.
